(19) United States
(12) Patent Application Publication
(10) Pub. No.: US 2016/0166436 A1
(43) Pub. Date: Jun. 16, 2016

(54) COVER AND SLEEVE PROTECTOR

(71) Applicant: Kenneth Hazelwood, Bryan, TX (US)

(72) Inventor: Kenneth Hazelwood, Bryan, TX (US)

(21) Appl. No.: 14/571,470

(22) Filed: Dec. 16, 2014

(51) Int. Cl. A61F 13/00; A61F 13/14

(52) U.S. Cl. CPC A61F 13/00038; A61F 13/00017; A61F 13/14; A61F 2013/00093; A61F 2013/00102; A61F 2013/00127; A61F 2013/00157

(57) ABSTRACT

A medical cover which includes: an elongated sleeve; a plastic impermeable surface; and a elastic seal at each end of the elongated sleeve, where the elastic seals prevent moisture from penetrating into the interior of the elongated sleeve. In one particular embodiment the elongated sleeve may include a single open end and a single elastic seal at the open end of the elongated sleeve, where the elastic seals prevent moisture from penetrating into the interior of the elongated sleeve. The elongated sleeve may be shaped like a sock for use on a foot of the user. In another alternative embodiment, the elongated sleeve includes sufficient volume to enclose the torso of a user.

### BACKGROUND OF THE INVENTION

1. Field of Invention

The present invention relates to a cover and sleeve protector that covers wounds, stitches, burns and IV connections to prevent damage from moisture.

2. Description of Related Art

Many injured individuals may have open wounds, stitches, burns and bandages and must keep these bandages, wounds or stitches away from moisture and water especially while taking a shower or bath. If the wound or injured area is exposed to moisture it could further damage or delay the healing process. One of the common methods used to protect a wounded area is the use of a plastic bag with tape to cover the injured area and therefore limit exposure to moisture while taking a shower or a bath. This solution however is sometimes ineffective because it is usually a high risk that the plastic bag may come unraveled or moisture will still leak into the bag therefore exposing the wound, stitch or injury to moisture. Therefore it would be advantageous to have a cover that is specifically designed to protect a wounded area from moisture while taking a shower or exposure to other moisture hazards.

### SUMMARY OF THE INVENTION

The present invention relates to a medical cover comprising: an elongated sleeve; a plastic impermeable surface; and a elastic seal at each end of the elongated sleeve, where the elastic seals prevent moisture from penetrating into the interior of the elongated sleeve. In one particular embodiment the elongated sleeve may include a single open end and a single elastic seal at the open end of the elongated sleeve, where the elastic seals prevent moisture from penetrating into the interior of the elongated sleeve. The elongated sleeve may be shaped like a sock for use on a foot of the user. In another alternative embodiment, the elongated sleeve includes sufficient volume to enclose the torso of a user.

### BRIEF DESCRIPTIONS OF DRAWINGS

FIG. 1 depicts a first embodiment of a medical cover in accordance with the present invention.

FIG. 2 depicts a second embodiment of a medical cover in accordance with the present invention.

FIG. 3 depicts a third embodiment of a medical cover in accordance with the present invention.

FIG. 4 depicts a fourth embodiment of a medical cover in accordance with the present invention.

### DETAILED DESCRIPTION

The present invention relates to a medical cover that is used to cover an injury such as a stitch, bandage, plaster, cast, IV and other wound or dressing that may be normally used on injuries. The importance of covering many of these injuries is imperative because the patient must not expose the injury to moisture. However many times these dressings or stitches are required to be in place for a number of days if not weeks and therefore it would be advantageous to have a particular cover specifically designed to protect the injury from moisture exposure.

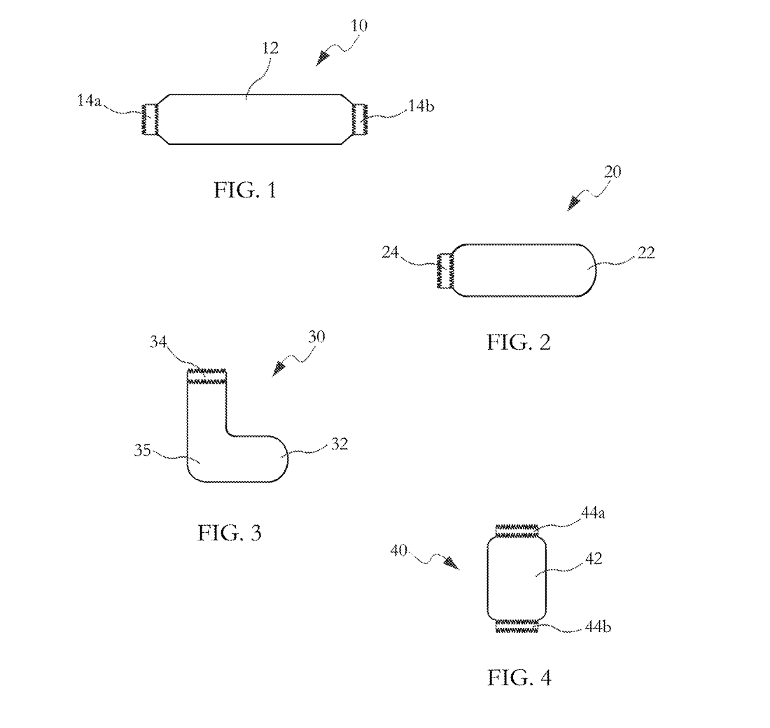

In reference to FIG. 1, a first embodiment of a Medical Cover 10 is depicted. The Medical Cover 10 includes an elongated Sleeve 12. This elongated body includes a plastic material surface that provides sufficient protection from moisture. During use the user may place the Sleeve 12 over an injury or a targeted area. At each end of the Sleeve 12 are an elastic seals 14a, 14b. These elastic seals seal the Sleeve 12 into place. The Medical Cover 10 therefore is simply slid over an appendage that may have an injury and each end is sealed through the elastic seals provided at each end of the Sleeve 12.

FIG. 2 depicts an alternative embodiment of the present invention. A Medical Cover 20 is depicted with a single open that includes an elastic seal. A Cuff 24 provides a seal for the Body Portion 22 of the Medical Cover 20. The Body Portion 22 includes a plastic surface that protects the underlying skin or injury from moisture exposure. Further in reference to FIG. 3 a third embodiment of the present invention is depicted. This third embodiment is Medical Cover 30 that is shaped in a form of a sock. As noted the sock has a Toe Portion 32 and Heel 35. At the entry point of the sock an Elastic Cuff 34 is provided. Again this Cuff 34 provides an elastic seal gripping the skin of the user and therefore preventing the entry of moisture into the interior of the sock. The Medical Cover 30 therefore provides an alternative embodiment for use on a individual's foot.

In reference to FIG. 4, a fourth embodiment of the present invention is depicted showing a Medical Cover 40. The Medical Cover 40 is similar to the Medical Cover 10 of FIG. 1 however the Body Portion 42 is expanded for use over a patient's torso. A top Opening 44a and bottom Opening 44b are provided so that the Medical Cover 40 may be slid over the torso of a user. Again at the top end 44a an elastic seal or cuff is provided that seals the interior of the Body Portion 42 similarly the bottom Opening 44b is also provided with such an elastic seal. These forms of medical covers therefore provide multiple versions and configurations for use on a patient. Each version is easily placed over a user's injury and provides an effective and sufficient cover for the user's wound or injury. The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A medical cover comprising:
a. an elongated sleeve;
b. a plastic impermeable surface; and
c. an elastic seal at each end of the elongated sleeve, where the elastic seals prevent moisture from penetrating into the interior of the elongated sleeve.

2. A medical cover comprising:
a. an elongated sleeve, where the elongated sleeve includes one open end;
b. a plastic impermeable surface; and
c. an elastic seal at the open end of the elongated sleeve, where the elastic seals prevent moisture from penetrating into the interior of the elongated sleeve.

3. The medical cover according to claim 2, where the elongated sleeve is shaped like a sock.

4. The medical cover according to claim 1, where the elongated sleeve includes sufficient volume to enclose the torso of a user.
